(19) United States
(12) Patent Application Publication
(10) Pub. No.: US 2025/0349107 A1
(43) Pub. Date: Nov. 13, 2025

(54) METHOD FOR EVALUATION OF CO2 PLUME DISTRIBUTION PATTERN BASED ON IMAGE SPATIAL MOMENT THEORY

(71) Applicant: CHINA UNIVERSITY OF PETROLEUM (EAST CHINA), Dongying (CN)

(72) Inventors: Shuyang LIU, Dongying (CN); Zhiqiang WANG, Dongying (CN); Hangyu LI, Dongying (CN); Junrong LIU, Dongying (CN); Baojiang SUN, Dongying (CN); Qizhi TAN, Dongying (CN); Rongjiang LIU, Dongying (CN); Wenyue SUN, Dongying (CN); Jianchun XU, Dongying (CN)

(21) Appl. No.: 19/060,670

(22) Filed: Feb. 22, 2025

(30) Foreign Application Priority Data
May 13, 2024 (CN) 202410590658.6

(51) Int. Cl. G06V 10/764; G06V 10/26

(52) U.S. Cl. CPC G06V 10/764; G06V 10/26

(57) ABSTRACT

The present disclosure discloses a method for the evaluation of CO₂ plume distribution pattern based on image spatial moment theory, which relates to the technical field of CO₂ geological storage in oil and gas reservoirs and saline aquifers, including: establishing a two-dimensional geological model for CO₂ storage in saline aquifers; generating a heterogeneous permeability model and calculating the corresponding heterogeneous capillary force coefficient; setting initial conditions of the model and control conditions for production wells and injection wells; studying the evolution characteristic of CO₂ plume during CO₂ injection and storage processes, and calculating the first-order and the second-order spatial moments of CO₂ plume images; dividing CO₂ distribution patterns based on CO₂ plume morphology; establishing a classification map for the distribution patterns of CO₂ plume; quantitatively evaluating the distribution patterns of the CO₂ plume.

### CROSS-REFERENCE TO RELATED APPLICATIONS

This application claims priority to Chinese Application No. 202410590658.6, filed on May 13, 2024, entitled “METHOD FOR EVALUATION OF CO2 PLUME DISTRIBUTION PATTERN BASED ON IMAGE SPATIAL MOMENT THEORY”. These contents are hereby incorporated by reference.

### TECHNICAL FIELD

The present disclosure relates to the technical field of CO₂ geological storage in oil and gas reservoirs and saline aquifers, in particular to a method for evaluation of CO₂ plume distribution pattern based on image spatial moment theory.

### BACKGROUND

CO₂ geological storage technology is one of the important means to address climate change and has made significant progress in recent years. This technology reduces carbon emissions in the atmosphere by injecting captured CO₂ into underground oil reservoirs, gas reservoirs, or saline aquifers for storage. CO₂ geological storage not only helps to reduce greenhouse gas emissions, but also improves the recovery rate of oil and gas reservoirs, and extends the sustainable utilization period of energy resources. Deep saline aquifers have the advantages of wide distribution and large reserves, and are potential sites for large-scale CO₂ sequestration in the future.

In the process of CO₂ injection and storage, heterogeneity of reservoir permeability is one of the most important and fundamental geological features. It increases the complexity of internal fluid migration and has a significant impact on the distribution of CO₂ plume in saline aquifers. At the same time, it also reduces the accuracy of CO₂ storage capacity assessment in saline aquifers and increases the risk of CO₂ leakage through production wells or open faults. Therefore, it has important scientific and engineering significance to strengthen the detailed characterization of underground reservoir heterogeneity and master the plume migration and evolution characteristics of CO₂ sequestration in heterogeneous reservoirs.

At present, research on the impact of permeability heterogeneity on CO₂ geological storage mostly focuses on the characteristic of dissolution and convection of CO₂ plume in vertical direction, neglecting the influence of horizontal heterogeneity on the distribution and evolution of CO₂ plume. In addition, the distribution patterns of CO₂ plume are only “dispersive type”, “fingering type”, and “channeling type”. Through extensive research, it has been found that this classification method is not sufficient to fully summarize the distribution and migration characteristics of CO₂ plume, and the evaluation of each distribution pattern lacks precise quantitative standards.

Therefore, in view of the above technical issues, it is urgent to conduct a more detailed and comprehensive classification of CO₂ distribution patterns, establish a graph of CO₂ plume distribution patterns considering the influence of horizontal and vertical heterogeneous parameters, and select appropriate dimensionless parameters to establish quantitative standards for CO₂ plume distribution patterns.

### SUMMARY

To solve the above technical problems, the present disclosure discloses a method for evaluation of CO₂ plume distribution pattern based on image spatial moment theory. This method establishes a geological model of CO₂ storage in a two-dimensional heterogeneous saline aquifer, studies the migration characteristic of CO₂ plume, calculates the heterogeneity coefficient of capillary forces and the spatial moments of CO₂ plume images, and establishes a classification map of CO₂ plume distribution patterns. Based on this map, the distribution pattern of CO₂ plume can be quantitatively evaluated for any given heterogeneous permeability field, providing theoretical support for CO₂ storage potential estimation, CO₂ injection scheme site selection, and leakage risk assessment.

To achieve the above objectives, the present disclosure adopts the following technical solution:

A method for evaluation of CO₂ plume distribution pattern based on image spatial moment theory is provided, which includes the specific steps as following:
S1, establishing a geological model for CO₂ sequestration in a two-dimensional saline aquifer, and initializing model parameters;
S2, generating heterogeneous permeability models with different dimensionless horizontal correlation lengths and dimensionless vertical correlation lengths, and calculating corresponding heterogeneous capillary force coefficients;
S3, setting initial conditions of the model and control conditions of a production well and an injection well;
S4, studying the evolution characteristics of CO₂ plume during CO₂ injection and storage processes, and calculating a first-order spatial moment and a second-order spatial moment of CO₂ plume images;
S5, dividing CO₂ plume distribution patterns based on CO₂ plume morphology;
S6, establishing a classification map of the CO₂ plume distribution patterns based on the dimensionless horizontal correlation lengths, the dimensionless vertical correlation lengths, the heterogeneous capillary force coefficients, and spatial moments of CO₂ plume images;
S7, calculating the spatial moments of the CO₂ plume images during CO₂ injection and storage processes for any given heterogeneous permeability model to quantitatively evaluate a distribution pattern of the CO₂ plume.

Further, the geological model for CO₂ sequestration in a two-dimensional saline aquifer in step S1 includes a permeable saline aquifer, a non-permeable overlying layer, and a non-permeable underlying layer.

Further, in step S2, using sequential Gaussian simulation method to generate a two-dimensional heterogeneous permeability field, so that the logarithm 1 g (k) of the permeability follows a normal distribution, and an arithmetic mean of the permeability remains consistent;

The heterogeneous capillary force coefficient V_{DP} is:

$${V}_{D P} = \frac{{k}_{50} - {k}_{84.1}}{{k}_{50}} \qquad ( 1 )$$

In the formula, k is the permeability, and k₅₀ and k_{84.1} are the permeability values when cumulative probabilities are 50% and 84.1%, respectively.

Further, in step S3, the initial conditions of the model and the control conditions of the production well and the injection well include: model size, depth, porosity, permeability, initial pressure, CO₂ injection rate of the injection well, and pressure of the production well.

Further, in step S4, the first-order spatial moment Rᵢ and the second-order spatial moment S of the CO₂ plume images are:

$${R}_{i} = \frac{1}{{M}_{0}} \int {x}_{i} ϕ ( x ) c ( x , t ) dx \qquad ( 2 )$$

$$S = \frac{1}{{M}_{0}} \int ( {x}_{i} - {R}_{i} ( t ) ) ( {x}_{j} - {R}_{j} ( t ) ) ϕ ( x ) c ( x , t ) dx \qquad ( 3 )$$

In the formula, M₀=∫ϕ(x)c(x,t)dx is the zero order spatial moment of the CO₂ plume images; x is the position; t is time; ϕ(x) is the porosity at the position where CO₂ is located, c(x,t) is the saturation of CO₂; xᵢ and xⱼ are the position coordinates in horizontal and vertical directions, respectively; and Rᵢ(t) and Rⱼ(t) are the first-order spatial moments in the horizontal and vertical directions, respectively.

Further, in step S5, the CO₂ plume distribution patterns include dispersive type, sweeping type, fingering type, and channeling type.

Further, in step S6, the classification map of the CO₂ plume distribution patterns is determined by the second-order spatial moment of the CO₂ plume images and the distribution morphology of the CO₂ plume.

The advantageous effect of the present disclosure is that the method for evaluation of the distribution pattern of CO₂ plume in heterogeneous saline aquifers can be adapted to various scenarios under different properties of reservoirs and initial conditions, clarify the migration and evolution characteristics of CO₂ plume in heterogeneous reservoirs, quantitatively evaluate the distribution pattern of CO₂ plume in permeability heterogeneous reservoirs, and have the advantages of simple operation and high accuracy. In addition, this method can quantitatively evaluate the distribution pattern of CO₂ plume in heterogeneous reservoirs, providing theoretical support for CO₂ storage potential estimation, CO₂ injection site selection, and leakage risk assessment.

### BRIEF DESCRIPTION OF THE DRAWINGS

FIG. 1 is a flowchart of the present disclosure;

FIG. 2 shows the geological model and grid division diagram of CO₂ sequestration in a two-dimensional heterogeneous saline aquifer in an application example of the present disclosure;

FIG. 3A shows the permeability field when the dimensionless horizontal and vertical correlation lengths are both 0.5 in an application example of the present disclosure;

FIG. 3B shows the normal distribution when the dimensionless horizontal and vertical correlation lengths are both 0.5 in the application example of the present disclosure;

FIG. 4A shows the first-order spatial moment of the CO₂ plume images when the dimensionless horizontal and vertical correlation lengths are both 0.5 in the application example of the present disclosure;

FIG. 4B shows the CO₂ plume images and the second-order spatial moment when the dimensionless horizontal and vertical correlation lengths are both 0.5 in the application example of the present disclosure;

FIG. 5 shows four modes of CO₂ plume distribution in heterogeneous reservoirs in the application example of the present disclosure;

FIG. 6 is a classification map of CO₂ plume distribution patterns in heterogeneous reservoirs in the application example of the present disclosure.

### DETAILED DESCRIPTION OF THE EMBODIMENTS

In order to make the technical problems, technical solutions and beneficial effects of the present invention clearer, the present invention will be further described in detail below with reference to the accompanying drawings and embodiments.

It should be understood that these embodiments are only used to illustrate the present invention, but the present invention is not limited thereto. In addition, it should be understood that after reading the content described in the present invention, those skilled in the art can make various changes or modifications to the present invention, and these equivalent technical means also fall within the scope of protection of the present invention.

In the present disclosure, a geological model construction module is used to establish a two-dimensional heterogeneous geological model and initialize model parameters; a calculation module is used to calculate the heterogeneous capillary force coefficient and the first-order and second-order spatial moments of CO₂ plume images; and a classification map construction module for CO₂ plume distribution patterns is used to establish a plume distribution pattern for evaluating the permeability heterogeneous saline aquifers for CO₂ sequestration. Establish a two-dimensional heterogeneous geological model and initialize model parameters, including: establishing a two-dimensional heterogeneous geological model for CO₂ sequestration in saline aquifers, including permeable saline aquifers, non-permeable overlying layers, and non-permeable underlying layers; dimensionless horizontal correlation length and dimensionless vertical correlation length; model size, depth, porosity, permeability, initial pressure, CO₂ injection rate of the injection well, and pressure of the production well; heterogeneous coefficient and spatial moment parameters, including: heterogeneous capillary force coefficient, the zero order spatial moment, the first-order spatial moment, and the second-order spatial moment of CO₂ plume images; Establish a classification map for the distribution patterns of CO₂ plume, including: the dimensionless horizontal correlation length, the dimensionless vertical correlation length, the heterogeneous capillary force coefficient, and the second-order spatial moments of CO₂ plume images.

### EMBODIMENT

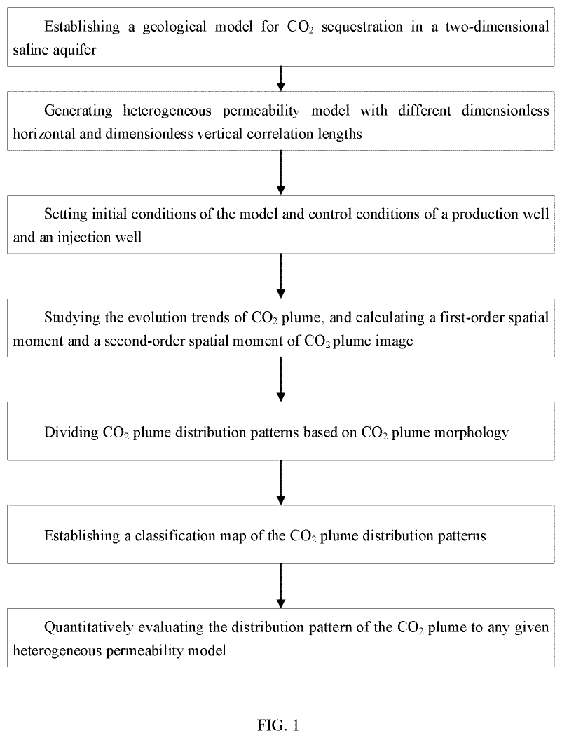

A method for evaluation of CO₂ plume distribution pattern based on image spatial moment theory is provided, and the evaluation process is shown in FIG. 1, including the following steps:

(1) Establish a geological model for CO₂ sequestration in a two-dimensional saline aquifer, and initialize model parameters, wherein the geological model for CO₂ sequestration in the two-dimensional saline aquifer includes a permeable saline aquifer, a non-permeable overlying layer, and a non-permeable underlying layer.

(2) Generate heterogeneous permeability models with different dimensionless horizontal correlation lengths and dimensionless vertical correlation lengths, and calculate corresponding heterogeneous capillary force coefficients.

The sequential Gaussian simulation method (SGSIM) is used to generate a two-dimensional heterogeneous permeability field, so that the logarithm of permeability with a base of 10 follows a normal distribution, and the arithmetic mean of the permeability remains consistent. Set the dimensionless horizontal and vertical correlation lengths to 0, 0.1, 0.5, and 0.9 respectively, but only consider the case where the dimensionless horizontal correlation length λₓ is greater than the dimensionless vertical correlation length λₓ.

The heterogeneous capillary force coefficient V_{DP} is:

$${V}_{D P} = \frac{{k}_{50} - {k}_{84.1}}{{k}_{50}} \qquad ( 1 )$$

In the formula, k is the permeability, and k₅₀ and k_{84.1} are the permeability values when cumulative probabilities are 50% and 84.1%, respectively.

(3) Set initial conditions of the model and control conditions of a production well and an injection well;

The initial conditions of the model and the control conditions of the production well and the injection well mainly include: model size, depth, porosity, permeability, initial pressure, CO₂ injection rate of the injection well, and pressure of the production well.

(4) Study the evolution characteristic of CO₂ plume during CO₂ injection and storage processes, and calculate the first-order spatial moment and the second-order spatial moment of CO₂ plume images.

The first-order spatial moment Rᵢ and the second-order spatial moment S of the CO₂ plume images are:

$${R}_{i} = \frac{1}{{M}_{0}} \int {x}_{i} ϕ ( x ) c ( x , t ) dx \qquad ( 2 )$$

$$S = \frac{1}{{M}_{0}} \int ( {x}_{i} - {R}_{i} ( t ) ) ( {x}_{j} - {R}_{j} ( t ) ) ϕ ( x ) c ( x , t ) dx \qquad ( 3 )$$

In the formula, M₀=∫ϕ(x)c(x,t)dx is the zero order spatial moment of the CO₂ plume images; x is the position; t is time; ϕ(x) is porosity at the position where CO₂ is located, c(x,t) is the saturation of CO₂; xᵢ and xⱼ are the position coordinates in horizontal and vertical directions, respectively; and Rᵢ(t) and Rⱼ(t) are the first-order spatial moments in the horizontal and vertical directions, respectively.

(5) Divide CO₂ plume distribution patterns based on CO₂ plume morphology.

The CO₂ plume distribution patterns include four types, which are dispersive type, sweeping type, fingering type, and channeling type.

(6) Establish a classification map of the CO₂ plume distribution patterns based on the dimensionless horizontal correlation lengths, the dimensionless vertical correlation lengths, the heterogeneous capillary force coefficients, and spatial moments of CO₂ plume images.

The classification map of the CO₂ plume distribution patterns is determined by the second-order spatial moment of the CO₂ plume images and distribution morphology of the CO₂ plume.

(7) For any given heterogeneous permeability model, calculate the spatial moments of the plume images during CO₂ injection and storage processes to quantitatively evaluate a distribution pattern of the CO₂ plume.

### Application Example

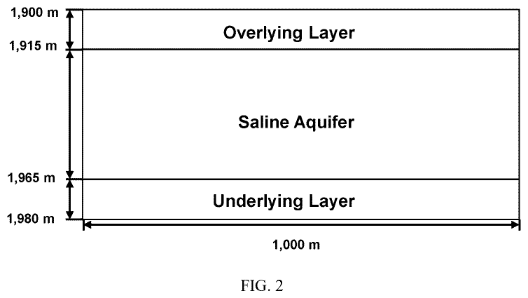

Based on the establishment of a two-dimensional Cartesian coordinate geological model, this study investigates the migration and evolution characteristics of CO₂ plume in heterogeneous saline aquifers. A method for evaluation of CO₂ plume distribution pattern based on image spatial moment theory is proposed, combined with FIG. 2 to FIG. 6. The specific steps are as follows:

(1) A two-dimensional Cartesian coordinate geological model is established using numerical simulation software CMG-GEM, which is divided into an upper non-permeable overlying layer, a middle heterogeneous permeable saline aquifer, and a lower non-permeable underlying layer, as shown in FIG. 2. The top depth of the model is 1900 m, the length of the model is 1000 m, and the total thickness of the model is 80 m. In addition, the thickness of the overlying and underlying layers is 15 m, the thickness of the middle saline aquifer is 50 m, and the number of evenly divided grids is 100×160 (x×z).

(2) Set the dimensionless horizontal and vertical correlation lengths to 0, 0.1, 0.5, and 0.9 respectively, but only consider the case where the dimensionless horizontal correlation length λₓ is greater than the dimensionless vertical correlation length λ_{z}.

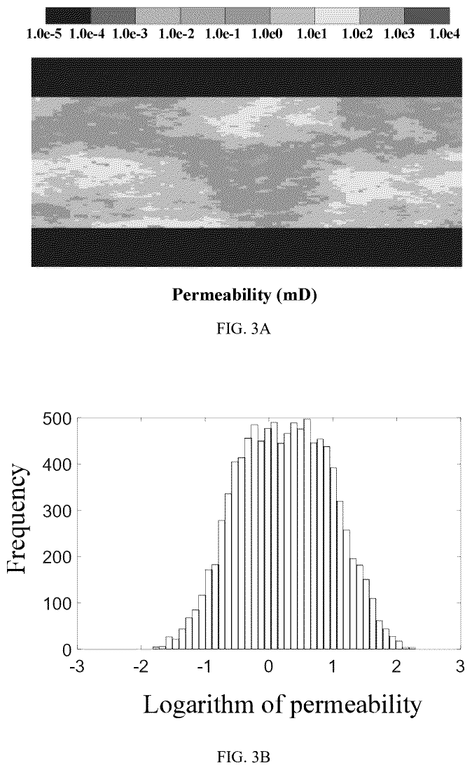

Generate a two-dimensional heterogeneous permeability field using the Sequential Gaussian Simulation Method (SGSIM) in SGeMS software, ensuring that the logarithm of permeability with a base of 10 follows a normal distribution, and the arithmetic mean of permeability for each case is 1 mD. When the dimensionless horizontal correlation length λₓ and the dimensionless vertical correlation length λ_{z} are both 0.5, the generated heterogeneous permeability field is shown in FIG. 3A, and the logarithm of permeability follows a normal distribution, as shown in FIG. 3B. The heterogeneous capillary force coefficient V_{DP} is 0.5636.

(3) Initialize the geological model, with the porosity of 0.01 and the permeability of 1.0×10⁻⁵ mD for both the overlying and underlying layers. The porosity of the middle saline aquifer is 0.3. The top interface pressure is 20 MPa, and the model temperature is 60° C. The CO₂ injection rate of the injection well is 2000 m³/d, the pressure of the production well is set to 20 MPa, and the model is initially saturated with saline water. After 10 years of CO₂ injection, the injection is stopped, and then the simulation continues for 90 years.

(4) Use CMG-GEM software to simulate the migration and evolution dynamics of CO₂ plume, and calculate the first-order spatial moment and the second-order spatial moment of CO₂ plume images based on CO₂ saturation distribution data and formulas (2) and (3).

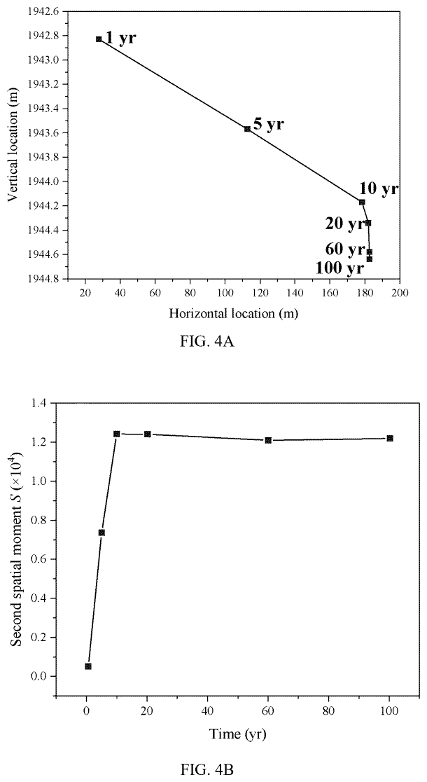

When the dimensionless horizontal correlation length λₓ and dimensionless vertical correlation length, are both 0.5, the variation of the first-order spatial moment with time is shown in FIG. 4A, and the variation of the second-order spatial moment with time is shown in FIG. 4B.

(5) Draw the CO₂ saturation distribution maps of each case at the time of simulated 100 years, as shown in FIG. 5. It can be seen that the distribution of CO₂ plume clearly presents four types: “dispersive type”, “sweeping type”, “fingering type”, and “channeling type”. Moreover, the second-order spatial moment can be used as a criterion for dividing distribution patterns: when the second-order spatial moment S<1.0×10⁴, the CO₂ plume is “dispersive type”; when the second-order spatial moment is 1.0×10⁴<S<1.9×10⁴, the CO₂ plume is “sweeping type”; when the second-order spatial moment is 1.9×10⁴<S<4.0×10⁴, the CO₂ plume is of the “fingering type”; and when the second-order spatial moment S>4.0×10⁴, the CO₂ plume is “channeling type”.

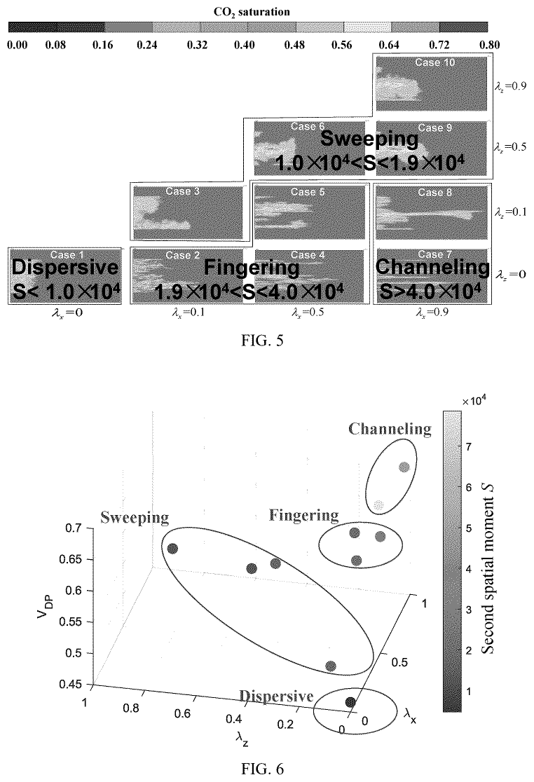

(6) Based on the dimensionless horizontal correlation lengths, the dimensionless vertical correlation lengths, the heterogeneous capillary force coefficients, and the spatial moments of CO₂ plume images, a classification map of CO₂ plume distribution patterns is established, as shown in FIG. 6. Note that the spatial moment used here is the second-order spatial moment of the CO₂ plume images.

(7) For any other given heterogeneous permeability model, determine the dimensionless horizontal correlation length, the dimensionless vertical correlation length, and the heterogeneous capillary force coefficient, after the initial and boundary conditions are given, the second-order spatial moment of the CO₂ plume images can be calculated based on the CO₂ saturation field data, and the distribution pattern of the CO₂ plume can be quantitatively evaluated based on the established classification map.

Certainly, the above descriptions are merely preferred embodiments of the present disclosure. The present disclosure is not limited to the above embodiments listed. It should be noted that, all equivalent replacements and obvious variations made by any person skilled in the art under the teaching of the specification fall within the essential scope of the specification and shall be protected by the present disclosure.

What is claimed is:

1. A method for evaluation of CO₂ plume distribution pattern based on image spatial moment theory, comprising:
S1, establishing a geological model for CO₂ sequestration in a two-dimensional saline aquifer, and initializing model parameters;
S2, generating heterogeneous permeability models with different dimensionless horizontal and dimensionless vertical correlation lengths, and calculating corresponding heterogeneous capillary force coefficients;
S3, setting initial conditions of the model and control conditions of a production well and an injection well;
S4, studying evolution characteristics of CO₂ plume during CO₂ injection and storage processes, and calculating a first-order spatial moment and a second-order spatial moment of CO₂ plume images;
S5, dividing CO₂ plume distribution patterns based on CO₂ plume morphology;
S6, establishing a classification map of the CO₂ plume distribution patterns based on the dimensionless horizontal correlation lengths, the dimensionless vertical correlation lengths, the heterogeneous capillary force coefficients, and spatial moments of CO₂ plume images;
S7, calculating the spatial moments of the CO₂ plume images during CO₂ injection and storage processes for any given heterogeneous permeability model to quantitatively evaluate a distribution pattern of the CO₂ plume.

2. The method for evaluation of CO₂ plume distribution pattern based on image spatial moment theory as claimed in claim 1, wherein the geological model for CO₂ sequestration in a two-dimensional saline aquifer in step S1 comprises a permeable saline aquifer, a non-permeable overlying layer, and a non-permeable underlying layer.

3. The method for evaluation of CO₂ plume distribution pattern based on image spatial moment theory as claimed in claim 2, wherein in step S2, using sequential Gaussian simulation method (SGSIM) to generate a two-dimensional heterogeneous permeability field, so that the logarithm of the permeability 1 g(k) follows a normal distribution, and an arithmetic mean of the permeability remains consistent;
the heterogeneous capillary force coefficient V_{DP} is:
$${V}_{D P} = \frac{{k}_{50} - {k}_{84.1}}{{k}_{50}} ;$$
in the formula, k is the permeability, and k₅₀ and k_{84.1} are the permeability values when cumulative probabilities are 50% and 84.1%, respectively.

4. The method for evaluation of CO₂ plume distribution pattern based on image spatial moment theory as claimed in claim 3, wherein in step S3, the initial conditions of the model and the control conditions of the production well and the injection well comprise: model size, depth, porosity, permeability, initial pressure, CO₂ injection rate of the injection well, and pressure of the production well.

5. The method for evaluation of CO₂ plume distribution pattern based on image spatial moment theory as claimed in claim 4, wherein in step S4, the first-order spatial moment Rᵢ and the second-order spatial moment S of the CO₂ plume images are:
$${R}_{i} = \frac{1}{{M}_{0}} \int {x}_{i} ϕ ( x ) c ( x , t ) dx ; \\ S = \frac{1}{{M}_{0}} \int ( {x}_{i} - {R}_{i} ( t ) ) ( {x}_{j} - {R}_{j} ( t ) ) ϕ ( x ) c ( x , t ) dx ;$$
in the formula, M₀=∫ϕ(x)c(x,t)dx is a zero order spatial moment of the CO₂ plume images; x is a position; t is time; ϕ(x) is porosity at the position where CO₂ is located, c(x,t) is saturation of CO₂; xᵢ and xⱼ are position coordinates in horizontal and vertical directions, respectively; and Rᵢ(t) and Rⱼ(t) are the first-order spatial moments in the horizontal and vertical directions, respectively.

6. The method for evaluation of CO₂ plume distribution pattern based on image spatial moment theory as claimed in claim 5, wherein in step S5, the CO₂ plume distribution patterns comprise dispersive type, sweeping type, fingering type, and channeling type.

7. The method for evaluation of CO₂ plume distribution pattern based on image spatial moment theory as claimed in claim 6, wherein in step S6, the classification map of the CO₂ plume distribution patterns is determined by the second-order spatial moment of the CO₂ plume images and distribution morphology of the CO₂ plume.
